(19) United States
(12) Patent Application Publication
(10) Pub. No.: US 2024/0389887 A1
(43) Pub. Date: Nov. 28, 2024

(54) SYSTEMS AND METHODS FOR MONITORING UPPER LIMB FUNCTION DURING ACTIVITIES OF DAILY LIVING

(71) Applicant: BioSensics LLC, Newton, MA (US)

(72) Inventors: Adonay Nunes Sastre, Quincy, MA (US); Ilkay Yildiz Potter, Fullerton, CA (US); Ram Kinker Mishra, Brighton, MA (US); Ashkan Vaziri, Newton, MA (US)

(21) Appl. No.: 18/672,415

(22) Filed: May 23, 2024

Related U.S. Application Data
(60) Provisional application No. 63/503,797, filed on May 23, 2023.

(51) Int. Cl. A61B 5/11; A61B 5/00

(52) U.S. Cl. CPC A61B 5/1124; A61B 5/1123; A61B 5/6824; A61B 5/7253; A61B 5/7267; A61B 2562/0219

(57) ABSTRACT

Systems and methods for monitoring limb function. An example method includes obtaining sensor data from individual devices worn on individual limbs of a user, the devices generating sensor data indicative of, at least, acceleration information associated with the limbs. The obtained sensor data is adjusted for input into a machine learning model, with the machine learning model being a deep learning model. A forward pass is computed through the machine learning model, with the machine learning model being trained to output information indicative of goal-directed movements (GDMs) performed by the user. Information indicative of GDMs is obtained via the machine learning model, with the information reflects particular labels identifying particular GDMs.

### CROSS-REFERENCE TO RELATED APPLICATIONS

This application claims priority to U.S. Prov. App. No. 63/503,797 titled “A WEARABLE SENSOR FOR MONITORING UPPER LIMB FUNCTION DURING ACTIVITIES OF DAILY LIVING” and filed on May 23, 2023, the disclosure of which is hereby incorporated herein by reference in its entirety.

### GOVERNMENT LICENSE RIGHTS

This invention was made with government support under Grant No. HD084035 awarded by the National Institutes of Health. The government has certain rights in the invention.

### BACKGROUND

### Technical Field

The present disclosure relates generally to wearable devices. Specifically, the present disclosure relates to wearable sensors for monitoring limbs.

### Description of Related Art

Objective and quantitative assessment of lower and upper limb movement and functions can facilitate early detection, disease progression monitoring, and development of personalized treatment plans for individuals with neurological disorders. Goal-directed movements (GDMs) are the atomic components of upper limb movements, and the movement patterns depend on the planned motor commands with hand trajectories toward specific target locations. In goal-directed movements, the central nervous system (CNS) coordinates between multiple muscle groups to work together in a specific sequence and timing to achieve a desired outcome. The CNS receives sensory information about the goal and the environment and uses this information to plan and execute the movement. This process involves several stages, including sensory processing, motor planning, motor programming, and execution.

GDMs are a crucial aspect of daily life for carrying out tasks such as reaching, grasping, and manipulating objects. Stroke can adversely affect goal-directed movement in multiple ways, including motor impairments, sensory processing deficits, and cognitive deficits. In stroke rehabilitation, remote and quantitative assessment of GDMs is essential for the clinicians to assess the patient's progress towards achieving functional goals.

At present, technical techniques to monitor neurological movements are inaccurate. In this example, prior techniques do not provide sufficient technical accuracy to inform quality or complexity of movements being performed.

### SUMMARY

The disclosed technology may be embodied in a computer-implemented method, system, and computer storage media. An example method includes obtaining sensor data from individual devices worn on individual limbs of a user, the devices generating sensor data indicative of, at least, acceleration information associated with the limbs; adjusting the obtained sensor data for input into a machine learning model, wherein the machine learning model is a deep learning model; computing a forward pass through the machine learning model, wherein the machine learning model is trained to output information indicative of goal-directed movements (GDMs) performed by the user; and obtaining, via the machine learning model, the information indicative of GDMs, wherein the information reflects particular labels identifying particular GDMs.

### BRIEF DESCRIPTION OF THE DRAWINGS

FIG. 1 illustrates a block diagram of an example movement analysis system.

FIG. 2 illustrates the area under the receiver operating characteristic curve (ROC-AUC) for differentiating goal-directed (GD) from non-GD movements.

FIGS. 3A-3B illustrate example coefficients according to the techniques described herein.

FIG. 4 is a flowchart of an example process to determine goal-directed movements (GDMs) based on input sensor information.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

This application describes an automated technique to detect goal-directed movements (GDMs) from limb-worn devices (e.g., wrist-worn) that obtain sensor data (e.g., accelerometer data). As will be described, machine learning techniques (e.g., neural networks, such as shallow or deep learning models) may be used to analyze the sensor data. Advantageously, the techniques described herein may utilize the sensor data to detect daily activities of an upper limb. Prior techniques relied upon physiological signal measurements (e.g., electromyography), and failed to detect GDMs. These prior techniques therefore failed to monitor upper limb activity, and therefore are not suitable for certain medical applications (e.g., stroke rehabilitation).

A system described herein (e.g., the movement analysis system 100) may receive sensor data and compute a forward pass through one or more machine learning models. As will be described, an example machine learning model may include a transformer-based neural network (e.g., transformer layers optionally with fully-connected layer(s) after the transformer layers). The example machine learning model may be trained to output information classifying, or otherwise identifying, GDMs based on input sensor data. The input sensor data may be adjusted prior to input into the machine learning model. For example, windows of input sensor data may be used where each window is a threshold length (e.g., 2 seconds, 3 seconds, 10 seconds). As another example, input sensor data may be normalized or certain values removed (e.g., values greater than, or less than, a threshold, such as an overall threshold or a threshold based on an average, moving average, median, and so on).

The system may represent, in some embodiments, a wrist-worn device which includes one or more processors to analyze input sensor data. For example, the wrist-worn device may include one or more inertial measurement units (IMUs). In some embodiments, a user may wear two wrist-worn devices. For example, two sensor data streams may be analyzed to inform detection of GDMs. As one example, the sensor data streams may represent multivariate time series (e.g., multi-channel accelerometer measurements. Similar to the above, individual windows may be extracted from the time series. For example, a time-period may be extracted as a window from a first wrist-worn device and the same time-period may be extracted as a window from a second wrist-worn device. The windows may be aggregated and provided, or individually provided, as input to the machine learning model. The system may additionally train the machine learning model and the wrist-worn device(s) may execute the machine learning models. In some embodiments, the wrist-worn devices may provide sensor data to the system which then executes the machine learning models.

In classifying windowed accelerometer data as GDM or non-GDM, the disclosed technology demonstrates that a state-of-the-art deep learning model outperforms existing shallow models designed for stroke rehabilitation applications. The model performance described herein, as one example, achieved an AUC of 0.9, sensitivity 0.81, specificity 0.84 and F1 0.82, outperforming existing shallow models.

In some embodiments, the techniques described herein may be used to inform additional information. For example, features may be extracted from GDM periods and used to predict whether the measurements were collected from a stroke survivor or a negative control participant. In addition, the features may also be used to predict the Fugl-Meyer Assessment (FMA) score (e.g., a stroke-specific performance-based impairment index) based on stroke survivors. The prediction performance was compared to the performance from models that used the entire recordings to extract the features, rather than only GDM periods.

The techniques described herein have important technological implications for the field of rehabilitation, as automatic detection of GDMs can be used to monitor patients' progress and provide feedback to clinicians. While previous techniques have used various algorithms to detect upper limb function, the disclosed technology leverages state-of-the-art deep learning models. Thus, the disclosed technology is an innovative approach to specifically detecting GDMs. Compared to prior techniques, the disclosed technology achieves more accurate results. For example, a prior technique reported an accuracy of 88% in controls and 70% in stroke survivors for detecting three types of arm movements using accelerometer data, while a second prior technique reported an accuracy of 84.5% for detecting hand gestures using electromyography and accelerometer data.

The system described herein may, as described above, extract features from detected GDM periods. Specifically, the system may use the features to predict whether a user is a stroke survivor or a control participant and to predict the Fugl-Meyer Assessment (FMA) score, which is a stroke-specific performance-based impairment index. The disclosed technology (e.g., a machine learning model) trained on features from GDM periods may outperform, in some embodiments, the model trained on features from the entire recording. Thus, the windowing described herein may be advantageous. Furthermore, the performance of regression of FMA scores may also be higher when using GDM features. These results suggest that not only can the disclosed technology accurately detect GDMs, but also the features extracted from these movements have additional information that can be used to differentiate between stroke survivors and control participants and to predict stroke-specific impairment. Example features for classification and regression may be related to zero crossings and indicate movement discontinuities with acceleration or velocity changing directions.

The disclosed technology shows that a deep learning model can achieve high levels of accuracy for automatic detection of goal-directed movements, even with data collected from stroke survivors, and suggests that deep learning models are a good candidate for monitoring upper limb function using limb-worn (e.g., wrist-worn) accelerometers. These results have important implications for the field of rehabilitation given the potential for the disclosed technology to be used as a valuable tool for stroke rehabilitation and could be extrapolated to monitoring populations with difficulties in upper limb function, such as in neurodegeneration.

This application describes improvements to prior techniques and addresses technological shortcomings associated with the prior techniques. Accelerometers provide a convenient way to measure physical activity and can be used to measure different movement patterns by detecting changes in acceleration. Previous methods relied on activity counts to measure upper limb function, usually by counting the number of zero crossings in the acceleration signal. However, activity counts provide an overall measure of physical activity and movement but are unable to differentiate between purposeful and non-purposeful movements, and do not provide information about the quality or complexity of the movements being performed. In contrast, GDM specifically measures upper limb function. As known by those skilled in the art, GDM tasks can detect early changes in upper limb function in neurodegenerative diseases and can be used to track disease progression over time. Accelerometer data can provide a cost-effective, and accurate, solution to measure GDMs and these findings highlight the importance of assessing GDM when measuring upper limb function in neurodegenerative diseases.

Automated assessments of GDM have advantages over manual assessments, including reducing the potential for human error and subjectivity, offering more frequent and convenient assessments, tracking disease progression, detecting subtle changes in movement, and being more cost-effective. These benefits are particularly important for neurorehabilitation settings where precise and reliable neuromotor assessments are critical for patient outcomes, and for conditions such as Parkinson's disease and ALS where frequent and accurate assessments can help with identifying changes in function and inform treatment decisions.

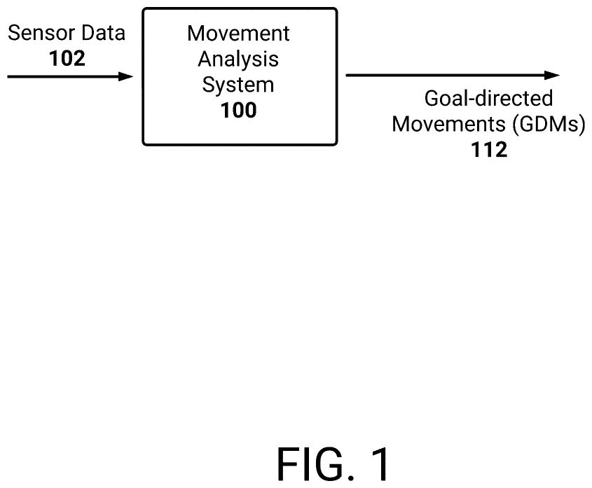

FIG. 1 illustrates an example movement analysis system 100. As described herein, the system 100 may be a system of one or more processors. For example, the system may be a cloud system which receives sensor data 102 from disparate devices (e.g., wrist-worn devices) worn by end-users. As another example, the system may be a mobile device associated with a user which is in communication (e.g., wireless communication) with one or more devices worn by the user. As another example, the system may represent a device worn by a user. For this example, the system may optionally receive information from a second device worn by the user. As an example, the user may have a device on each wrist.

In the illustrated example, the movement analysis system 100 is receiving sensor data 102. As described herein, the sensor data may represent information from an inertial measurement unit (IMU), such as a six-axis IMU. The sensor data 102 may include accelerometer information associated with movement of a user's limb (e.g., an arm). The system 100 may execute one or more machine learning models as described herein and output information indicating goal-directed movements (GDMs). The information may indicate whether a portion of the input sensor data 102 is indicative of, or otherwise classified as, a GDM or a non-GDM. The information may additionally indicate types of GDMs as described below.

In some embodiments, the output information (e.g., GDMs 112) may be presented via a user interface. For example, in embodiments in which the system 100 is a wearable device, a display of the wearable device may output a user interface identifying GDMs in substantially real-time. As another example, a mobile device (e.g., a smart phone) in communication with a wearable device may output GDMs in substantially real-time or present historical information. Similarly, the system 100 may present a user interface on an end-user device.

As an example of training, training data may be generated based on monitoring users and/or persons recruited for training purposes. For example, a portion of training data was prepared using 30 participants, from which 20 were stroke survivors (age mean 54.4, SD 10.1, time since stroke mean 4.6, SD 5.5, FMA average 37, SD 8), and 10 controls (age mean 53.8, SD 11.4).

Persons or users may be outfit with IMUs included in wearable devices, such as six-axis inertial measurement units (IMUs) on each wrist. Participants may perform example tasks while sensor data from the wearable devices is generated. Resulting study data may include tasks resembling different types of activities of daily living (ADL). Specifically, participants may perform unimanual, bimanual and passive tasks. These tasks may include the specific GDMs which are being identified and may include the tasks described in Table 1 below. Some stroke survivors performed a subset of the tasks based on their motor capabilities as assessed by a therapist. Each motor task was repeated a threshold number of times (e.g., 3 times) in order to capture intra-subject variability. A specialist, or automated system, may script and time the tasks. Except for the passive tasks, the tasks may be performed seated in an armless chair in front of a table. The walking task may be performed in a designated area with a tape indicating the beginning and end, subjects were instructed to walk a threshold number of laps (e.g., 1 lap, 2 laps, 3 laps, 5 laps). The system 100 may receive the sensor data (e.g., via substantially real-time streaming or as a file) at a particular sampling rate (e.g., 128 Hz, 256 Hz, 512 Hz, 644 Hz).

Description of the tasks for each type of movement performed while accelerometer data was recorded.

TABLE 1

| Type | Task |
|---|---|
| Unimanual | Drink from a can |
| (affected limb) | Turn a key in a lock |
| | Hair brushing |
| Bimanual | Pick up pen from desk, remove |
| | the cap, and place it back |
| | Pick up a box and bring it to the |
| | knees |
| | Fold a hand towel |
| Passive | Walk |
| | Stand up without using arm for |
| | bracing |
| | Ascend and descend stairs |
| Task Free | Periods without goal directed |
| | movements while accelerometer |
| | data is recorded |

Training data may be manually labeled or automatically labeled. For example, sensor data may be labeled via the sensor data in individual windows (e.g., individual segments). In this example, video or images of the persons may be analyzed to determine appropriate labels (e.g., analyzed via an automated machine learning model or human labeled). Each segment may be labeled, for example, as unimanual, bimanual, passive or task-free. During unimanual movements, the opposite side sensor data may be labeled as task-free (e.g., opposite limb). For the GD detection task, bimanual and active-side unimanual may be categorized as GD movements and the passive-side unimanual, passive and task-free may be categorized as non-GD.

The above-described six-axis IMU may include an accelerometer that detects acceleration and a gyroscope that measures rotation. In some embodiments, only accelerometer data may be used (e.g., to preserve battery life of a wearable device, to better ensure long-term monitoring). In some embodiments, accelerometer and rotation information may be used.

In some embodiments, the system 100 may band-pass acceleration time series data (e.g., triaxial sensor data) for each wrist IMU between a threshold range (e.g., 0.1 and 12 Hz optionally with a 4th order filter to remove the inertial gravity component and high frequency activity), and then downsampled to a threshold sampling rate (e.g., 25 Hz). Triaxial velocity data may be estimated by the system via integrating acceleration data. Then, the same band-pass filter may be applied. High frequency cut-off may advantageously not discard any activity measurements, as typical movements may be, for example, at most a threshold frequency (e.g., 10 Hz).

The system 100 may apply a sliding window of a threshold length (e.g., 2 seconds, 3 seconds, 5 seconds) with a particular overlap (e.g., 70% overlap). Thus, the system 100 may segment the data for training the machine learning model. At each window, if a threshold percentage (e.g., one third) of the time points were labeled as GD, the entire window was labeled as GD movement. For each IMU (e.g., on the same person), data may be windowed separately. In total, and as one example of training data, 49,254 6-dimensional time windows were extracted, with 21% labeled as GD. Optimal window size and overlap were determined from validation set performance, as explained below.

In some embodiments, a leave-one-subject-out (LOSO) cross-validation may be used to test machine learning model performance. A threshold percentage (e.g., 10%) of the training subjects for each cross-validation split may be further held-out as validation data and remaining training subjects may be used for training. The system may perform data normalization, such as via subtracting the population mean from each sample and dividing the resulting values by the population standard deviation, where mean and standard deviations were estimated from the training set for each split.

If the distribution of GD vs. non-GD windows is imbalanced, the training objective for each class may be weighted with the ratio of the other class in training. The validation set may be used for early-stopping of model training and for finding the optimal probability threshold to differentiate the two classes. The optimal threshold may be found by taking the geometric mean of the true positive rate and the true negative rate.

The system 100 may train, and execute, disparate classifiers for multivariate time-series signals to differentiate GD activity windows from others. As one example, a decision-tree classifier using gradient boosting termed XGBoost may be used. XGBoost was designed particularly to tackle small and imbalanced datasets via ensembling and pruning. Moreover, this classifier outperformed other non-deep learning models in multivariate time-series classification tasks, including measuring activities of daily living, and attained comparable performance to deep learning.

Another example classifier may be a transformer model (e.g., an attention-based neural network which leverages one or more transformer layers). To initialize the transformer classifier weights, an autoencoder model including a transformer encoder and a fully-connected decoder may be trained over the multivariate time-series samples in the training data. For example, the system 100 may minimize a masked reconstruction error loss in an unsupervised manner. The transformer encoder architecture may optionally use modifications of fully-trainable positional encoding, batch normalization, and so on. Following unsupervised pre-training, the decoder may be replaced by a fully-connected layer with a scalar output and sigmoid activation. The resulting transformer classifier may be fine-tuned by minimizing a cross-entropy loss to classify each input window as GD or non-GD.

Another example machine learning model may include a convolutional neural network, such as an explainable convolutional neural network. The machine learning model may aggregate features from 1D and 2D convolutions with model interpretability via gradient-weighted class activation mapping. Advantageously, this example machine learning model may be designed for multivariate time-series classification and has been shown to outperform other models when classifying physiological signals.

Output from these machine learning models are described in more detail below. The system 100 may optionally implement one, or all, of these machine learning models. In some embodiments, a user of the system 100 (e.g., a person wearing one or more wearable devices) may select the machine learning model to use.

As described above, in addition to classifying input sensor data 102 as being a GDM, or a specific GDM thereof as described above (e.g., in Table 1), or non-GDM, features from GD periods may be used to classify additional aspects. For example, the system may use features from inferred GD periods (e.g., from both limbs, such as hands) to classify stroke survivors. In addition, the system may analyze whether FMA can be better predicted with GD features (e.g., from individual windows) compared to features from an entire recording of sensor data (e.g., non-separated into windows). As an example, a threshold number of features (e.g., 14 features) were estimated from the acceleration and velocity time series sensor input. The time series were either tri-axial or magnitudes over the three axes. Tri-axial measures included the correlation between axis pairs and the number, mean length and length entropy of zero crossing segments. Measures based on magnitude were the minimum, maximum, median, root mean square, the domain frequency over energy (the peak frequency divided by the total spectrum), skewness, kurtosis, and entropy.

As an example of the training data used for experimental outputs below, 1830 activity periods were labeled, with 961 task-free, 79 passive, 249 unimanual and 541 bimanual activities. This corresponded to 608 hours of task-free activity, 44.5 hours of passive activity, 56 hours of unilateral movements and 138.5 hours of bilateral movements. The stroke participants represented 72.5% of the data. As only the affected side for stroke patients or the non-dominant side for controls was used during unimanual activities, the passive side was labeled as non-GD for these activities. When a sliding window of 3 seconds with a 70% overlap was applied, the total number of windowed time series used for training and testing were 49,254, out of which 21% were labeled as goal-directed movements. This includes both right and left side IMU accelerometer data.

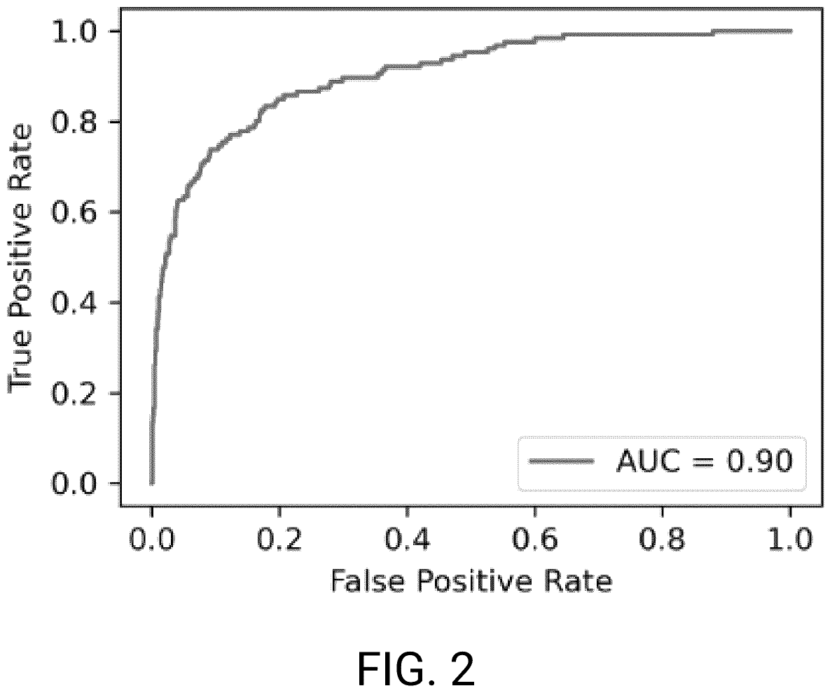

For the results below, a leave-one-subject-out (LOSO) cross validation was used to assess GD activity detection performances (e.g., accuracies) the above-described machine learning models (e.g., XGBoost, transformer-based model, explainable convolutional neural network such as an XCM model). The model performance was calculated over the test set of each cross validation split with respect to Area under the receiver operating characteristic (AUC), sensitivity, specificity and F1 score metrics. Average AUC, sensitivity, specificity and F1 score metrics for all methods are reported in Table 2 (below). XCM outperformed other shallow and deep models and attained 0.90 AUC, 0.81 sensitivity, 0.84 specificity and 0.82 F1 score. The receiver operating characteristic curve (ROC) for the XCM model is visualized in FIG. 2.

TABLE 2
GD activity detection performance

| Method | AUC | Sensitivity | Specificity | F1 score |
|---|---|---|---|---|
| XGBoost | 0.83 | 0.75 | 0.77 | 0.75 |
| Transformer | 0.83 | 0.78 | 0.75 | 0.76 |
| XCM | 0.90 | 0.81 | 0.84 | 0.82 |

Positive class, e.g., GD labels, may include an active hand (e.g., active limb) during unimodal movements and both hands during bimanual activities. Non-GD labels may include the passive hand (e.g., passive or non-active limb) during unilateral movements and passive activities such as walking and standing up from a chair without armrest. Non-GD labels may also include task-free periods in which subjects are not performing either task. To further analyze the performance of the XCM model during different tasks, the accuracy in different tasks was calculated separately for the stroke survivors and controls, with results presented in Table 3. For both groups, accuracy calculated over different sides were close to each other, showing promise for generalization performance of XCM.

Model performance for different tasks in stroke survivor and control groups.

TABLE 3

| Side | Bimanual | Unimanual | Passive | Task free |
|---|---|---|---|---|
| Stroke participants | | | | |
| affected | 0.81 | 0.82 | 0.83 | 0.79 |
| unaffected | 0.76 | 0.73 | 0.85 | 0.77 |
| Control participants | | | | |
| dominant | 0.94 | 0.89 | 0.99 | 0.81 |
| non-dominant | 0.91 | 0.76 | 1.0 | 0.83 |

The system 100 may train an elastic net logistic regression model to differentiate between stroke survivors and controls using features extracted solely from periods labeled as GDM (e.g., by a machine learning model, such as an XCM model) and using features extracted from the entire recording. As described herein, the entire recording may represent features from the above-described sensor data which are not separated into windows. A LOSO cross-validation was used, and the performance was assessed with average accuracy, sensitivity and specificity. The model using GDM features outperformed the setting of using entire recordings, with a balanced accuracy of 0.9, sensitivity of 0.1 and specificity of 0.8, compared to an accuracy of 0.75, sensitivity of 1 and specificity of 0.5. This indicates that features learned from GDM windows have more information to differentiate between groups, showing further promise for rehabilitation applications.

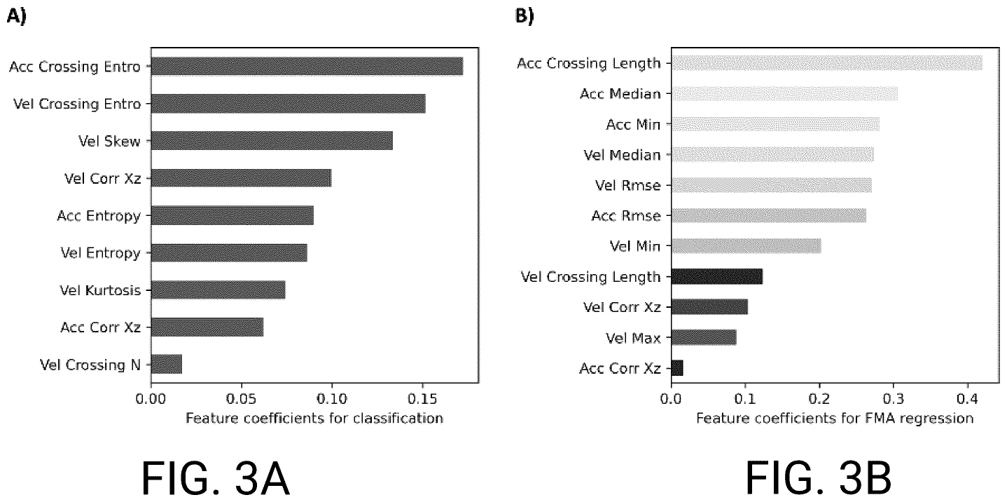

FIG. 3A shows example logistic regression coefficient magnitudes corresponding to features extracted from GDM periods that substantially contributed to classification. The regression performance for FMA scores was also higher when using GDM features, with a mean absolute error of 6.9 and an explained variance of 21%, compared to a mean absolute error of 8 and an explained variance of 0.6%. FIG. 3B shows the largest elastic net linear regression coefficient magnitudes, for example important features in predicting FMA scores. The extracted features from GDM may be highly sensitive in identifying stroke survivors from controls. Table 4 shows the group mean and standard deviation, the effect size and the p-value for the group differences significance.

In FIGS. 3A-3B, FIG. 3A: Feature coefficients for classifying stroke survivors from controls. The bar indicates that the mean in the stroke group is higher than controls. FIG. 3B: Feature coefficients for regression FMA, colors from yellow to dark blue indicate larger to smaller correlation between the feature and FMA scores. Acc=acceleration, vel=velocity, entro=entropy, N=number

TABLE 4

| | Stroke Survivors | | | Control Participants | | | | |
|---|---|---|---|---|---|---|---|---|
| Feature | Mean | ± | SD | Mean | ± | SD | Cohen's D | p-val |
| Acc min (m/s2) | 0.21 | ± | 0.1 | 0.5 | ± | 0.11 | −2.81 | <0.001 |
| Acc median (m/s2) | 2.04 | ± | 0.52 | 3.1 | ± | 0.39 | −2.2 | <0.001 |
| Acc RMS | 2.87 | ± | 0.58 | 4.14 | ± | 0.59 | −2.18 | <0.001 |
| Acc crossing entropy | 1.04 | ± | 0.02 | 1 | ± | 0.02 | 2.28 | <0.001 |
| Vel skewness | 0.86 | ± | 0.24 | 0.37 | ± | 0.11 | 2.34 | <0.001 |
| Vel crossing entropy | 1.07 | ± | 0.01 | 1.05 | ± | 0.01 | 2.3 | <0.001 |
| Vel median (m/s) | 31.79 | ± | 7.49 | 46.76 | ± | 5.82 | −2.14 | <0.001 |
| Acc entropy | 6.55 | ± | 0.44 | 5.87 | ± | 0.18 | 1.84 | <0.001 |
| Vel RMS | 41.76 | ± | 8.28 | 55.85 | ± | 6.45 | −1.82 | <0.001 |
| Vel entropy | 6.59 | ± | 0.46 | 5.89 | ± | 0.17 | 1.81 | <0.001 |
| Vel kurtosis | 0.53 | ± | 0.77 | −0.55 | ± | 0.17 | 1.69 | <0.001 |
| Vel crossing number (n) | 75.61 | ± | 43.6 | 32.13 | ± | 5.1 | 1.21 | 0.004 |
| Vel min | 4.21 | ± | 1.54 | 5.92 | ± | 1.13 | −1.2 | 0.004 |
| Acc crossing average | 8.82 | ± | 2.1 | 11.2 | ± | 2.12 | −1.13 | 0.007 |
| length (samples) | | | | | | | | |
| Acc crossing number (n) | 494.36 | ± | 376.97 | 159.91 | ± | 26.59 | 1.08 | 0.010 |
| Vel corr XZ | −0.02 | ± | 0.15 | −0.17 | ± | 0.14 | 1.02 | 0.014 |
| Vel max (m/s) | 96.22 | ± | 13.46 | 107.81 | ± | 11.65 | −0.9 | 0.028 |
| Acc corr XZ | −0.03 | ± | 0.09 | −0.1 | ± | 0.09 | 0.81 | 0.045 |
| Acc corr YZ | −0.31 | ± | 0.11 | −0.22 | ± | 0.12 | −0.81 | 0.046 |
| Acc max (m/s2) | 10.78 | ± | 2.09 | 12.47 | ± | 2.17 | −0.8 | 0.048 |
| Acc kurtosis | 5.81 | ± | 3.88 | 3.29 | ± | 1.6 | 0.76 | 0.060 |
| Acc skewness | 1.67 | ± | 0.46 | 1.38 | ± | 0.22 | 0.73 | 0.071 |
| Vel corr YZ | −0.55 | ± | 0.21 | −0.48 | ± | 0.19 | −0.37 | 0.347 |
| Acc dom freq over energy | 0.00013 | ± | 0.00015 | 9.37E−05 | ± | 2.78E−05 | 0.32 | 0.421 |
| Acc corr XY | 0.0137 | ± | 0.08 | 0.00078 | ± | 0.08 | 0.16 | 0.690 |
| Vel crossing average | 44.83 | ± | 2.82 | 45.27 | ± | 3.6 | −0.14 | 0.718 |
| length (samples) | | | | | | | | |
| Vel corr XY | 0.03 | ± | 0.13 | 0.01 | ± | 0.1 | 0.14 | 0.723 |
| Vel dom freq over energy | 5.60E−07 | ± | 1.87E−07 | 5.7E−07 | ± | 1.42E−07 | −0.07 | 0.860 |

Acc = acceleration,
Vel = velocity,
corr = correlation,
dom = domain,
freq = frequency

As described above, system 100 may train, and execute, machine learning models (e.g., the above-described three models) on accelerometer data to detect goal-directed movements during tasks resembling activities of daily living. Based on the results, for the training data used the best performing deep learning model achieved an AUC of 0.90. A prior technique which leveraged training on data from unimanual, bimanual and passive tasks using a Random Forest classifier achieved worse performance. The disclosed technology, such as via XCM, not only outperforms Random Forest in GDM detection with respect to three classification metrics (Table 2), but also does not discard task-free recordings. Thus, the disclosed technology has been trained on, and can attain, high accuracy over a wider range of ADL, and thus, is more generalizable to real-life applications.

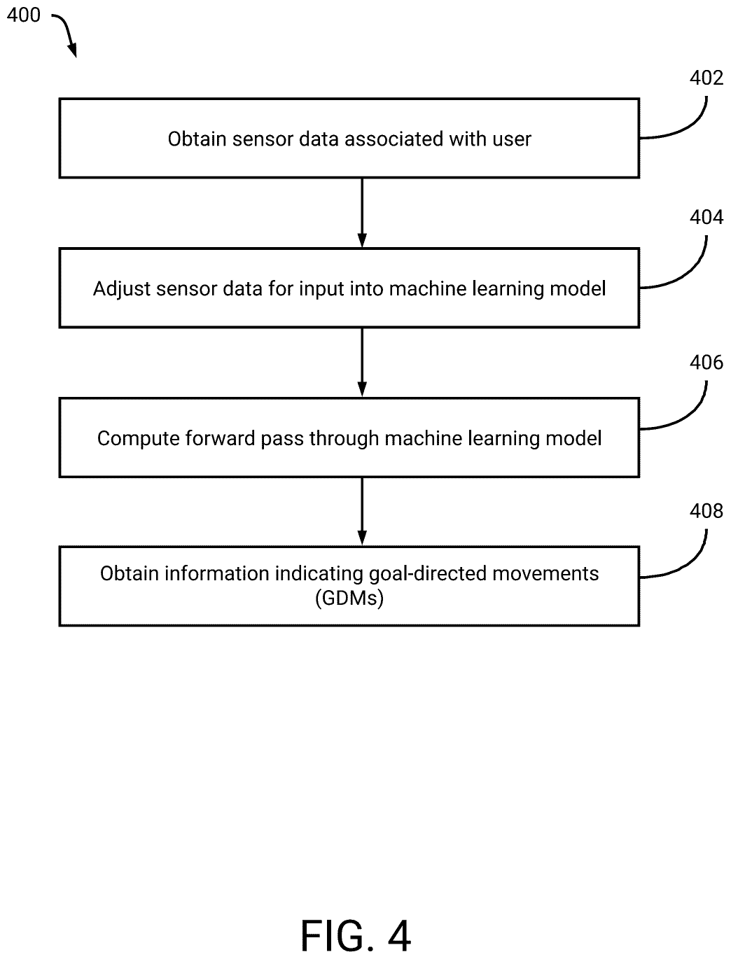

FIG. 4 is a flowchart of an example process 400 to determine goal-directed movements (GDMs) based on input sensor information. For convenience, the process 400 will be described as being performed by a system of one or more computers (e.g., the movement analysis system 100).

At block 402, the system obtains sensor data associated with a user. The system may obtain sensor data from wrist-worn or limb-worn devices on a user. In some embodiments, the user may wear one device on a particular wrist (e.g., a dominant hand). In some embodiments, the user may wear one device on each of the user's wrists. The sensor data may reflect information from an inertial measurement unit (IMU) included in each of the wearable devices, for example multi-axis accelerometer data (e.g., information in three perpendicular axes).

At block 404, the system adjusts the sensor data for input into a machine learning model. The system may normalize and/or filter the sensor data as described herein. Additionally, the system may separate the sensor data into individual windows of sensor data. For example, the windows may include values from the sensor data within a threshold period of time (e.g., values obtained at a particular sampling rate as described herein).

At block 406, the system computes a forward pass through the machine learning model. The machine learning models may include the example models described herein, for example a transformer-based model, an explainable convolutional neural network (XCM), and so on. Thus, the system may determine output for individual windows with the output reflecting information indicative of goal-directed movements (GDMs). For example, labels may indicate whether a window of time is associated with a GDM. Example labels are included above with respect to FIG. 1.

Examples of training the machine learning models are described above with respect to FIG. 1. For example, training data may be obtained based on monitoring participants performing example actions. Labels may be assigned to windows of sensor data and used to update weights forming layers of the models. The models may be trained to output the above-described labels or values indicative of the labels.

At block 408, the system obtains information indicating GDMs. As described above, the system may obtain labels or values indicative of GDMs based on output from the machine learning model.

In some embodiments, the system may refine the machine learning models based on the obtained sensor data. For example, the user may indicate (e.g., via verbal cues, textual input, input with a user interface, and so on) whether they were performing a GDM. In this example, the information may be used to update the weights of the machine learning model.

The system may aggregate GDMs for the user over time. For example, historical information may be generated. In this example, the system may present charts, summary information, and so on, which describe the aggregated GDMs. As an example, the system may present information indicative of particular GDMs performed at particular times, summaries of frequencies of GDMs, and so on.

### OTHER EMBODIMENTS

All of the processes described herein may be embodied in, and fully automated, via software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence or can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and engines described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might” or “may,” unless specifically stated otherwise, are understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

It should be emphasized that many variations and modifications may be made to the above-described embodiments, the elements of which are to be understood as being among other acceptable examples. All such modifications and variations are intended to be included herein within the scope of this disclosure.

What is claimed is:

1. A method implemented by a system of one or more processors, the system performing a focused assessment with sonography for trauma (FAST) exam, and the method comprising:
obtaining sensor data from individual devices worn on individual limbs of a user, the devices generating sensor data indicative of, at least, acceleration information associated with the limbs;
adjusting the obtained sensor data for input into a machine learning model, wherein the machine learning model is a deep learning model;
computing a forward pass through the machine learning model, wherein the machine learning model is trained to output information indicative of goal-directed movements (GDMs) performed by the user; and
obtaining, via the machine learning model, the information indicative of GDMs, wherein the information reflects particular labels identifying particular GDMs.

2. The method of claim 1, wherein the sensor data is generated via individual inertial measurement units (IMUs) included in individual devices, and wherein the acceleration information reflects tri-axis acceleration data.

3. The method of claim 1, wherein the individual devices include two devices worn on respective wrists of the user.

4. The method of claim 1, wherein adjusting the obtained sensor data comprises separating the sensor data into windows of sensor data, wherein individual windows are associated with a threshold amount of time.

5. The method of claim 1, wherein the information indicative of GDMs includes whether an individual window is associated with a GDM.

6. The method of claim 1, wherein the machine learning model is a transformer-based deep learning model.

7. The method of claim 1, wherein the machine learning model is an explainable convolutional neural network.

8. The method of claim 1, wherein the information indicative of GDMs includes labels identifying whether portions of the sensor data reflect unimanual actions, bimanual actions, passive actions, or are task-free.

9. The method of claim 1, further comprising causing presentation of an interactive user interface comprising summary information associated with the information indicative of GDMs.

10. A system comprising one or more processors and non-transitory computer storage media storing instructions that when executed by the one or more processors, cause the one or more processors to perform operations comprising:
obtaining sensor data from individual devices worn on individual limbs of a user, the devices generating sensor data indicative of, at least, acceleration information associated with the limbs;
adjusting the obtained sensor data for input into a machine learning model, wherein the machine learning model is a deep learning model;
computing a forward pass through the machine learning model, wherein the machine learning model is trained to output information indicative of goal-directed movements (GDMs) performed by the user; and
obtaining, via the machine learning model, the information indicative of GDMs, wherein the information reflects particular labels identifying particular GDMs.

11. The system of claim 10, wherein the sensor data is generated via individual inertial measurement units (IMUs) included in individual devices, and wherein the acceleration information reflects tri-axis acceleration data.

12. The system of claim 10, wherein the individual devices include two devices worn on respective wrists of the user.

13. The system of claim 10, wherein adjusting the obtained sensor data comprises separating the sensor data into windows of sensor data, wherein individual windows are associated with a threshold amount of time.

14. The system of claim 10, wherein the information indicative of GDMs includes whether an individual window is associated with a GDM.

15. The system of claim 10, wherein the machine learning model is a transformer-based deep learning model.

16. The system of claim 10, wherein the machine learning model is an explainable convolutional neural network.

17. The system of claim 10, wherein the information indicative of GDMs includes labels identifying whether portions of the sensor data reflect unimanual actions, bimanual actions, passive actions, or are task-free.

18. Non-transitory computer storage media storing instructions that when executed by a system of one or more processors, cause the processors to perform operations comprising:
obtaining sensor data from individual devices worn on individual limbs of a user, the devices generating sensor data indicative of, at least, acceleration information associated with the limbs;
adjusting the obtained sensor data for input into a machine learning model, wherein the machine learning model is a deep learning model;
computing a forward pass through the machine learning model, wherein the machine learning model is trained to output information indicative of goal-directed movements (GDMs) performed by the user; and
obtaining, via the machine learning model, the information indicative of GDMs, wherein the information reflects particular labels identifying particular GDMs.

19. The computer storage media of claim 18, wherein the machine learning model is a transformer-based deep learning model or an explainable convolutional neural network.

20. The computer storage media of claim 18, wherein the information indicative of GDMs includes labels identifying whether portions of the sensor data reflect unimanual actions, bimanual actions, passive actions, or are task-free.
